(19) United States
(12) Patent Application Publication
(10) Pub. No.: US 2018/0338711 A1
(43) Pub. Date: Nov. 29, 2018

(54) HEARING ASSESSMENT SYSTEMS AND RELATED METHODS

(71) Applicant: Mayo Foundation for Medical Education and Research, Rochester, MN (US)

(72) Inventors: David P. Argue, Rochester, MN (US); Alaa Koleilat, Northfield, MN (US); Stephen C. Ekker, Rochester, MN (US); Lisa A. Schimmenti, Orono, MN (US); Gayla L. Poling, Rochester, MN (US)

(21) Appl. No.: 15/987,063

(22) Filed: May 23, 2018

Related U.S. Application Data
(60) Provisional application No. 62/510,485, filed on May 24, 2017.

(51) Int. Cl. A61B 5/12; H04R 1/10; A61B 5/00

(52) U.S. Cl. CPC A61B 5/123; H04R 2460/01; A61B 5/6898; H04R 1/1091

(57) ABSTRACT

A method of executing a hearing test includes sending an audio signal to a noise cancelling audio device worn by a user, outputting instructions associated with the audio signal to a portable computing device having a display screen viewed by the user, receiving an input associated with audio signal via the display screen of the portable computing device, generating an audiogram based at least in part on the input, and outputting the audiogram to the portable computing device for display on the display screen to the user.

### CROSS-REFERENCE TO RELATED APPLICATIONS

This application claims the benefit of U.S. Provisional Application Ser. No. 62/510,485, filed May 24, 2017. The disclosure of the prior application is considered part of (and is incorporated by reference in) the disclosure of this application.

### TECHNICAL FIELD

This disclosure relates to hearing test systems, and more particularly to portable audiogram systems.

### BACKGROUND

Hearing loss is the most common sensory impairment in the U.S. and is a major factor in a variety of diseases. However, assessing hearing loss is a complex process and a logistical challenge for many candidates with hearing concerns. For example, obtaining an assessment can involve arranging an appointment with an audiologist, may not be covered by health insurance, and may be fatiguing and time consuming. Therefore, identifying and monitoring hearing loss in an efficient, flexible manner is an important, yet challenging endeavor.

### SUMMARY

The present disclosure provides a hearing assessment tool for performing a hearing test in a variety of settings (e.g., including non-clinical environments). The hearing assessment tool can allow hearing test candidates to conveniently check their hearing outside of a clinical setting and potentially avoid consuming resources (e.g., time, money, and transportation) that would otherwise be required to obtain a hearing test in a traditional clinical setting.

In one aspect, a method of executing a hearing test includes sending an audio signal to a noise cancelling audio device worn by a user, outputting instructions associated with the audio signal to a portable computing device having a display screen viewed by the user, receiving an input associated with audio signal via the display screen of the portable computing device, generating an audiogram based at least in part on the input, and outputting the audiogram to the portable computing device for display on the display screen to the user.

Embodiments can include one or more of the following features.

In some embodiments, sending the audio signal includes sending a sound at a particular frequency and duration.

In certain embodiments, the noise cancelling audio device is a set of portable noise cancelling headphones.

In some embodiments, the method further includes storing the audiogram in an electronic medical record.

In certain embodiments, the method includes generating a description of a hearing assessment result based at least in part on the input and outputting the description to the portable computing device for display on the display screen to the user.

In another aspect, a hearing assessment system includes a noise cancelling audio device configured to be worn by a user, a portable computing device having a display screen, and executable instructions that configure the portable computing device to perform a method. In some embodiments, the method includes sending a series of hearing assessment audio signals to the noise cancelling audio device, outputting, on the display screen, instructions associated with the series of hearing assessment audio signals, receiving inputs from the user in response to the series of hearing assessment audio signals, generating an audiogram based on the inputs, and outputting results of the audiogram on the display screen.

### DESCRIPTION OF DRAWINGS

FIG. 1 depicts a hearing assessment system that can execute implementations of the present disclosure.

FIG. 2 depicts a hearing assessment tool that can be implemented via the hearing assessment system of FIG. 1.

FIG. 3 depicts a user interface generated by the hearing assessment tool of FIG. 2.

FIG. 4 depicts a user interface generated by the hearing assessment tool of FIG. 2.

FIG. 5 depicts a user interface generated by the hearing assessment tool of FIG. 2.

FIG. 6 depicts a user interface generated by the hearing assessment tool of FIG. 2.

FIG. 7 depicts a user interface generated by the hearing assessment tool of FIG. 2.

FIG. 8 depicts a graph that shows an audiogram generated using the hearing assessment tool of FIG. 2 and an audiogram generated using a conventional hearing assessment tool.

FIG. 9 depicts a graph that shows an audiogram generated using the hearing assessment tool of FIG. 2 and an audiogram generated using a conventional hearing assessment tool.

FIG. 10 depicts an example process that can be executed via the hearing assessment tool of FIG. 2.

FIG. 11 depicts a graph that shows an audiogram (GA) generated using the hearing assessment tool of FIG. 2 and an audiogram (GS) generated using a conventional, gold standard hearing assessment tool for a subject's left ear.

FIG. 12 depicts a graph that shows an audiogram (GA) generated using the hearing assessment tool of FIG. 2 and an audiogram (GS) generated using a conventional, gold standard hearing assessment tool for a right ear of the subject of FIG. 11.

FIG. 13 depicts a graph that shows an audiogram (GoAudio) generated using the hearing assessment tool of FIG. 2 and an audiogram (Gold Standard) generated using a conventional, gold standard hearing assessment tool for a subject's left ear.

FIG. 14 depicts a graph that shows an audiogram (GoAudio) generated using the hearing assessment tool of FIG. 2 and an audiogram (Gold Standard) generated using a conventional, gold standard hearing assessment tool for a right ear of the subject of FIG. 13.

FIG. 15 depicts a graph that shows an audiogram (GoAudio) generated using the hearing assessment tool of FIG. 2 and an audiogram (Gold Standard) generated using a conventional, gold standard hearing assessment tool for a subject's left ear.

FIG. 16 depicts a graph that shows an audiogram (GoAudio) generated using the hearing assessment tool of FIG. 2 and an audiogram (Gold Standard) generated using a conventional, gold standard hearing assessment tool for a right ear of the subject of FIG. 15.

Like reference symbols in the various figures indicate like elements. In some examples, illustrations shown in the drawings may not be drawn to scale.

### DETAILED DESCRIPTION

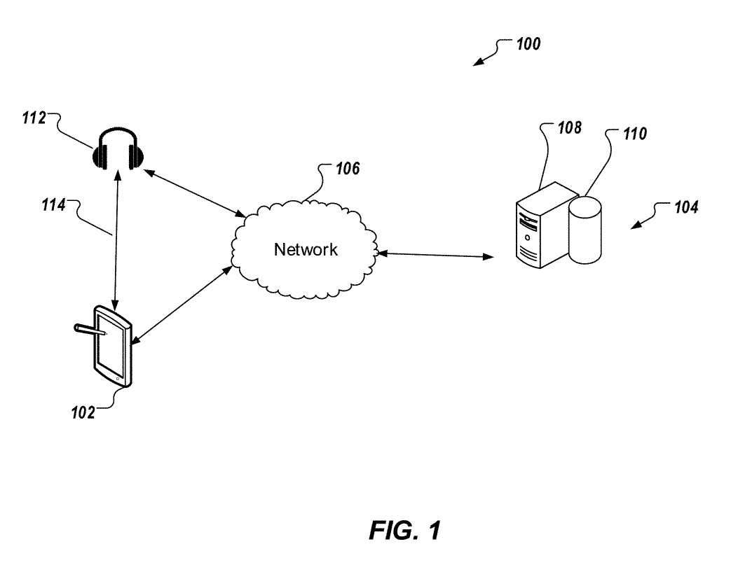

FIG. 1 depicts a hearing assessment system 100 that provides a mobile digital audiogram technology for performing a hearing test in a variety of settings (e.g., clinics, personal residences, and other non-clinical environments). The hearing assessment system 100 includes a computing device 102, an audio device 112, a server system 104, and a network 106. In the example of FIG. 1, the computing device 102 is embodied as a tablet computer. However, in some embodiments, the computing device 102 may be embodied as a different mobile data processing device, such as a laptop computer, a smart phone, or a tablet-based device. The audio device 112 is configured to cancel acoustic environmental noise to simulate environments (e.g., environments that may have maximum permissible noise at certain frequencies, as governed by associated standards) that are provided at clinical settings for performing hearing tests in a conventional manner. In this manner, the hearing assessment system 100 can enable high resolution hearing testing based on air-conduction hearing thresholds in any setting. In the example of FIG. 1, the audio device 112 is embodied as a set of noise cancelling headphones. However, in some embodiments, the audio device 112 may be embodied as another type of noise cancelling device, such as noise cancelling earplugs or other ear-level transducers.

The computing device 102 and the audio device 112 are portable devices that may communicate with the server system 104 over the network 106 and that can thereby interact with application software provided by the server system 104. The computing device 102 and the audio device 112 can communicate with each other wirelessly over the network 106 or directly via a wired connection 114. The computing device 102 and the audio device 112 are also blue-tooth enabled such that the devices 102, 112 can communication with each other wirelessly, directly without use of the network 106.

Generally, the server system 104 includes one or more computers in one or more physical locations. For example, the server system 104 includes a server 108 and a database 110. The server 108 may be of various forms including, but not limited to, a web server, an application server, a proxy server, a network server, or a server farm. For example, the server 108 may be an application server that executes software accessed by the computing device 102 and the audio device 112 via the network 106. A user (e.g., a clinician, a patient, an associate of the patient, or another hearing test administrator) can invoke applications available on the server 108 via a user-interface application (e.g., a web browser) running on the computing device 102. Each application can individually access data from the database 110.

In some embodiments, the hearing assessment system 100 is a distributed client/server system that spans one or more networks that may include the network 106. The network 106 can be a large computer network, such as a local area network (LAN), a wide area network (WAN), the Internet, a cellular network, or a combination thereof connecting any number of computing device 102 and server systems 104. In some embodiments, each computing device 102 can communicate with the server system 104 through a virtual private network (VPN), a Secure Shell (SSH) tunnel, or other secure network connection. In some embodiments, the network 106 can include the Internet, a wireless service network, or a Public Switched Telephone Network (PSTN). In some embodiments, the network 106 may include a corporate network (e.g., an intranet) and one or more wireless access points.

The computing device 102 provides a touchscreen that can present user interfaces that are generated by a hearing test software tool and that are configured to receive inputs from users of the software tool. The inputs are sent over the network 106 to the server system 104, on which the tool is executed. The server system 104 can store the inputs, generate outputs (e.g., user interfaces including graphical and textual outputs) based on the inputs, and send the outputs over the network 106 to the computing device 102, which can display the outputs to users of the software tool.

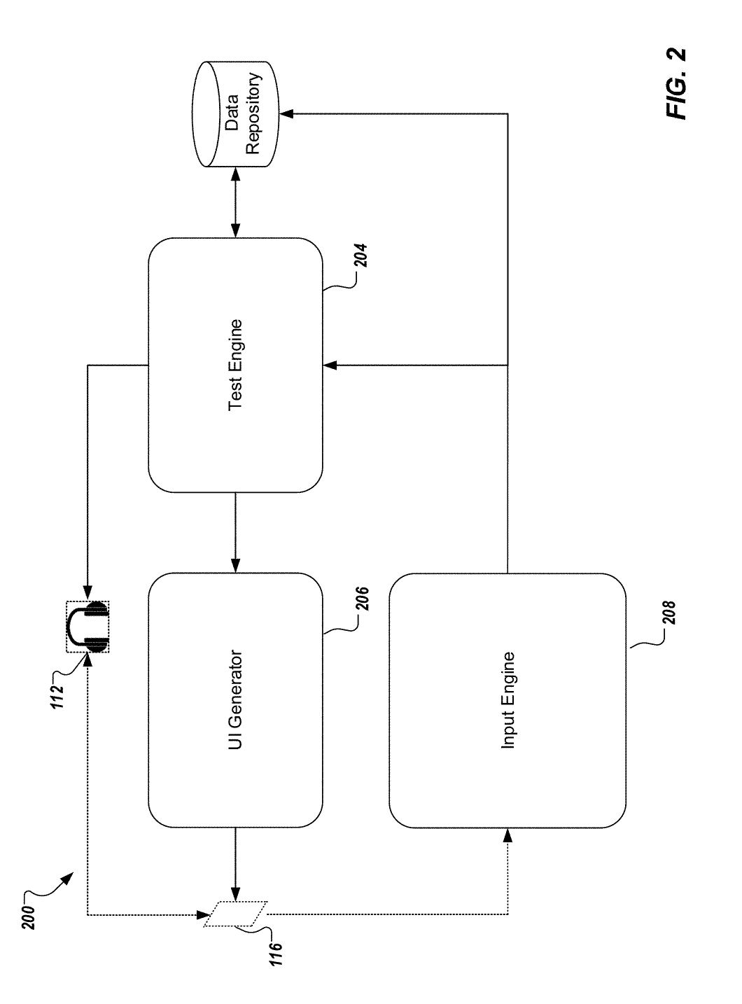

FIG. 2 depicts a hearing assessment tool 200 (e.g., a software tool) that is provided as a mobile computing application implemented on the computing device 102 of the hearing assessment system 100. Accordingly, the hearing assessment tool 200 can send audio signals to the audio device 112, generate audiograms for display on the computing device 102, generate user interfaces for display on the computing device 102, receive inputs at the computing device 102, and send data to the server system 104 for storage in the database 108. In some implementations, the hearing assessment tool 200 is called ‘Go!Audio.’

The hearing assessment tool 200 includes a data repository 202, a test engine 204, a user interface (UI) generator 206, and an input engine 208. The hearing assessment tool 200 can be downloaded as an app from the server system 104 and implemented on the computing device 102 and the database 110. For example, the data repository 202 is implemented on the database 110 and stores multiple hearing test algorithms for administration of hearing tests to patients, stores hearing test results, and stores user profile data. In some examples, the data repository 202 is implemented as an electronic medical records (EMR) database. The hearing test algorithms can include sound frequencies, amplitudes, associated durations (e.g., time periods), hearing thresholds, presentation levels, and reversals, etc. The test engine 204, the UI generator 206, and the input engine 208 are implemented on the computing device 102.

The test engine 204 generates audio signals according to a hearing test algorithm stored in the data repository 202 and sends the audio signals to the audio device 112 to execute a hearing test. The test engine 204 also sends an indication of the audio signals to the UI generator 206 for generation of associated user interfaces that are displayed to the user of the hearing assessment tool 200 on a touchscreen 116 of the computing device 102 while the audio signals are delivered to the audio device 112. The input engine 208 receives inputs at the user interfaces generated by the UI generator 206 and sends the inputs to the test engine 204 and/or the data repository 202. For example, the input engine 208 may receive an indication of when a patient first hears or can no longer hear a sound administered by the audio device 112 and may send the indication to the test engine 204 as an input to an audiogram that can be subsequently generated by the test engine 204. The audiogram can be further sent to the UI generator 206 for incorporation into a user interface output to the computing device 102 and/or sent to the data repository 202 for storage. In other cases, the input engine 208 may receive user login information and access user profile data stored in the data repository 202 to verify the user login information.

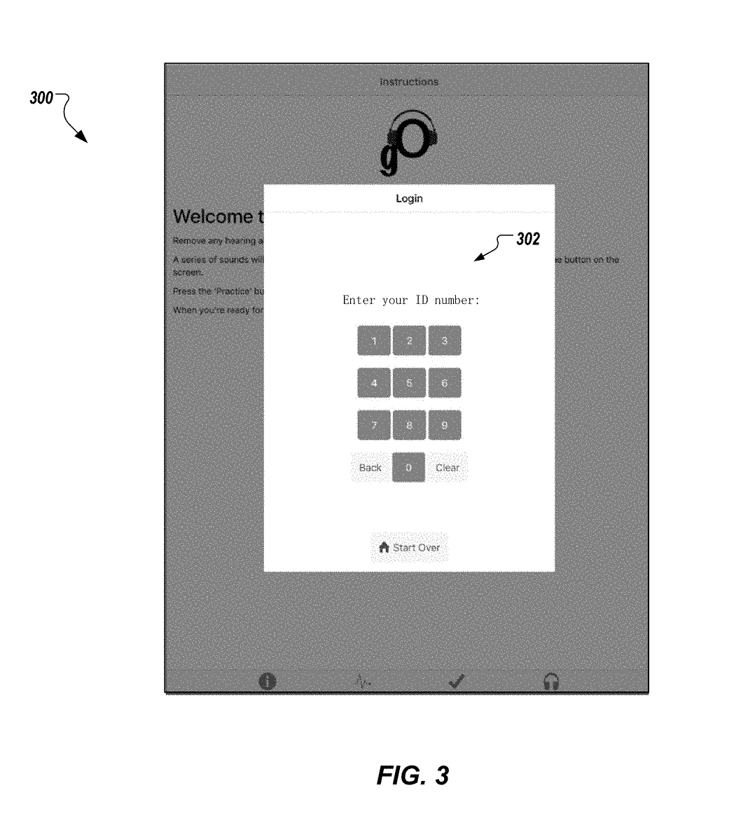

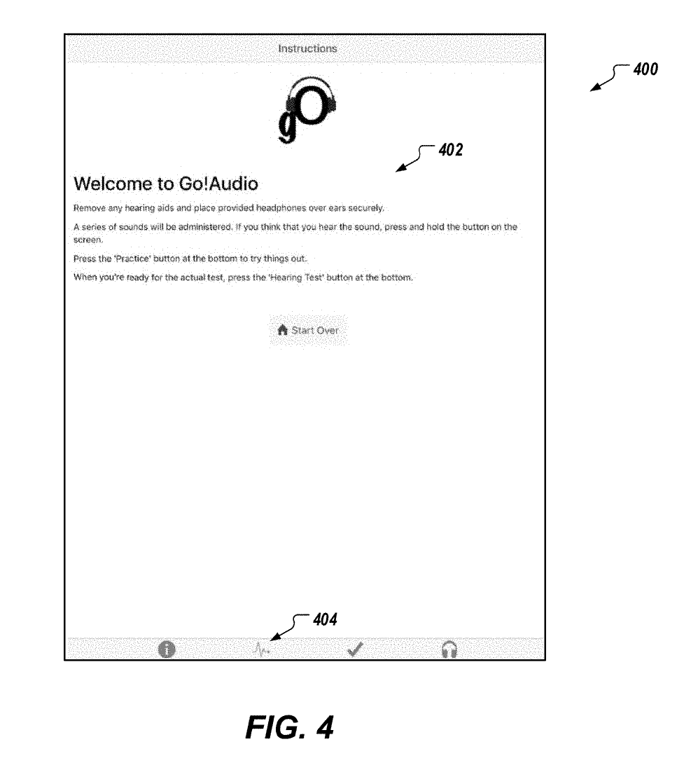

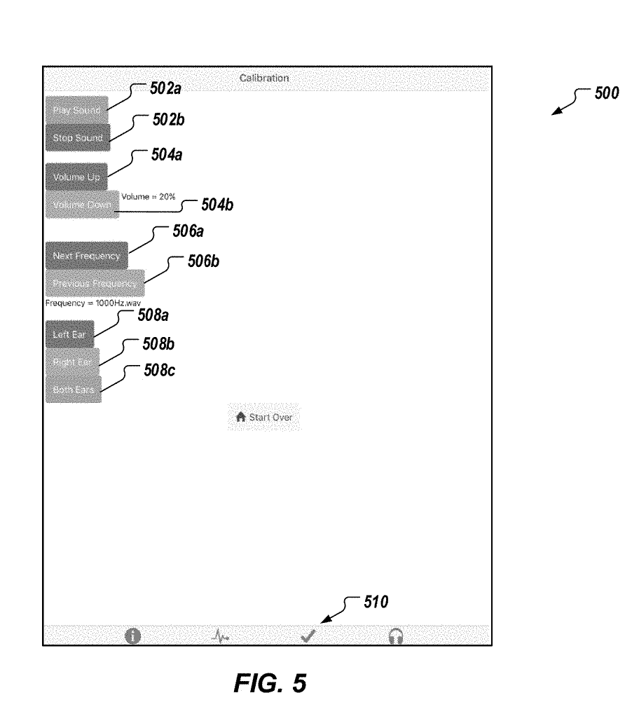

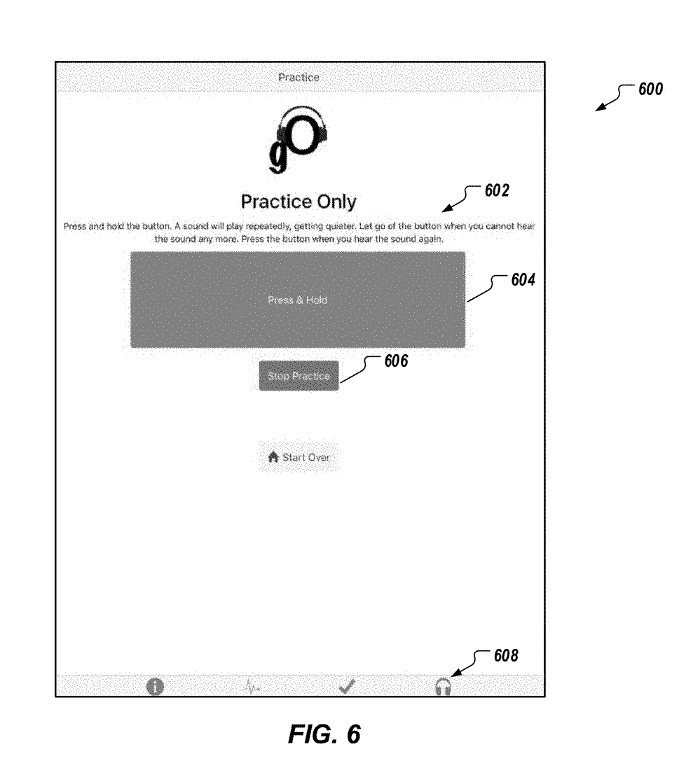

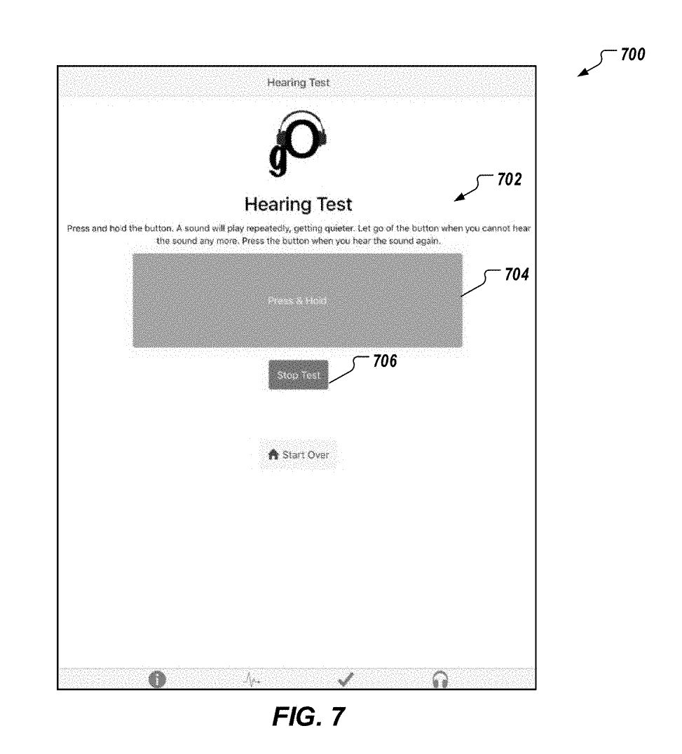

FIGS. 3-7 depict a series of user interfaces that can be generated and outputted to the computing device 102 by the UI generator 206. For example, FIG. 3 depicts a user interface 300 that provides a keypad 302 for accepting a user identification (ID) number that can be used to access the hearing assessment tool 200. Once the input engine 208 receives and verifies the user ID number, a user interface 400 is generated and outputted to the computing device 102. The user interface 400 provides instructions to a user (e.g., a patient) for using the hearing assessment tool 200 to carry out a hearing test. After the input engine 208 receives a selection of a Calibration icon 404, a user interface 500 is generated and outputted to the computing device 102. The user interface 500 provides selectors 502a, 502b for calibrating sound, selectors 504a, 504b for calibrating volume, selectors 506a, 506b for calibrating frequency, and selectors 508a, 508b, 508c for selecting one or both ears.

After the input engine 208 receives a selection of the Practice icon 510, the user interface 600 is generated and outputted to the computing device 102. The user interface 600 provides instructions 602, a button 604 for practicing the hearing test, and a button 606 for stopping the practice hearing test. After the input engine 208 receives a selection of the Hearing Test icon 608, the user interface 700 is generated and outputted to the computing device 102. The user interface 700 provides instructions 702, a button 704 for executing the hearing test, and a button 706 for stopping the hearing test. After the input engine 208 receives a selection of the button 706 for stopping the hearing test, the test engine 204 generates an audiogram based on the inputs received during the hearing test. The test engine 204 sends the audiogram to the data repository 202 for storage and to the UI generator 206 for incorporation into a display that is outputted to the computing device 102 for review by the user of the hearing test tool 200.

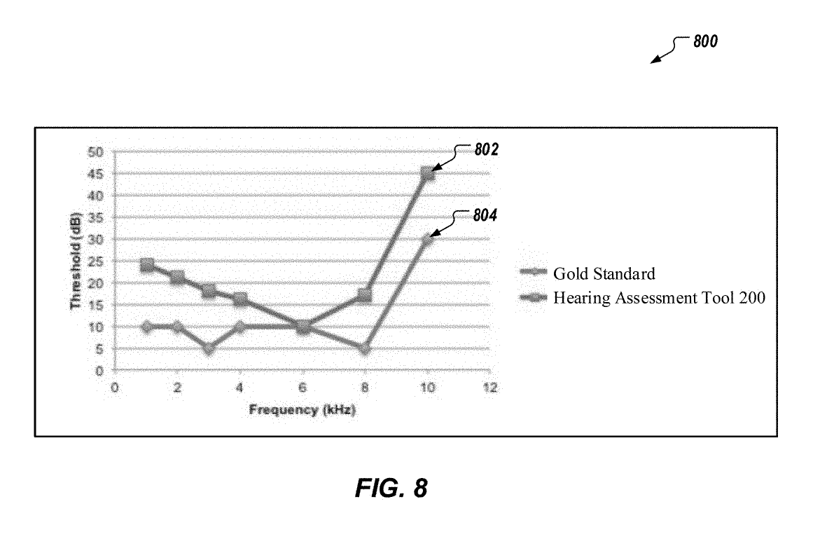

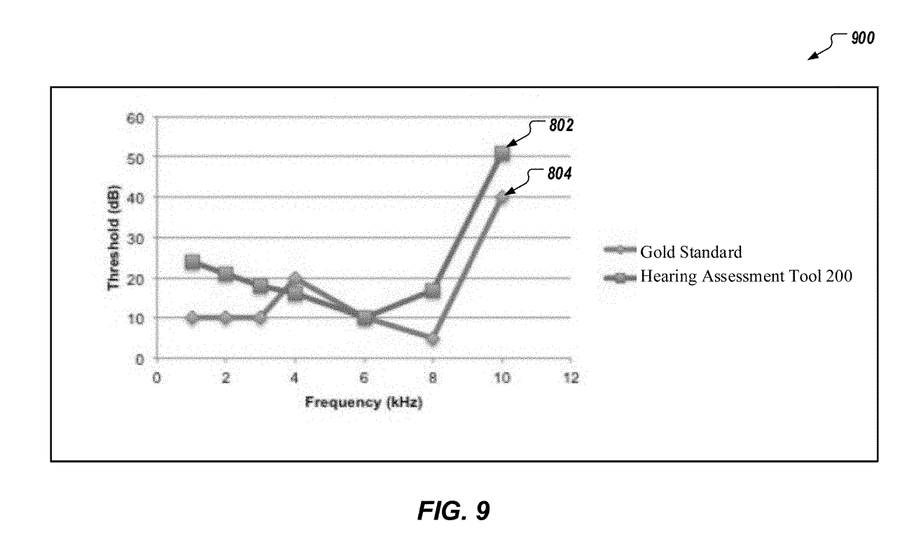

FIG. 8 depicts a graph 800 that shows an audiogram 802 (red) generated for a right ear of a patient using the hearing test tool 200 and an audiogram 804 (blue) generated for the right ear of the patient using a conventional, gold standard hearing test. Similarly, FIG. 9 depicts a graph 900 that shows an audiogram 902 (red) generated for a left ear of the patient using the hearing test tool 200 and an audiogram 904 (blue) generated for the left ear of the patient using the conventional, gold standard hearing test. This figure demonstrates that the hearing test tool 200 can be used to obtain comparable air-conducted hearing thresholds as compared to the gold standard hearing test. A normal/abnormal criteria of less than or equal to 25 dB HL can indicate normal hearing, and greater than 25 dB HL can indicate abnormal hearing. Based on the results, a message may be displayed to the user, indicating that further evaluation is or is not warranted, that the data may be stored, and/or that the data may be forwarded for review by an audiologist is desired.

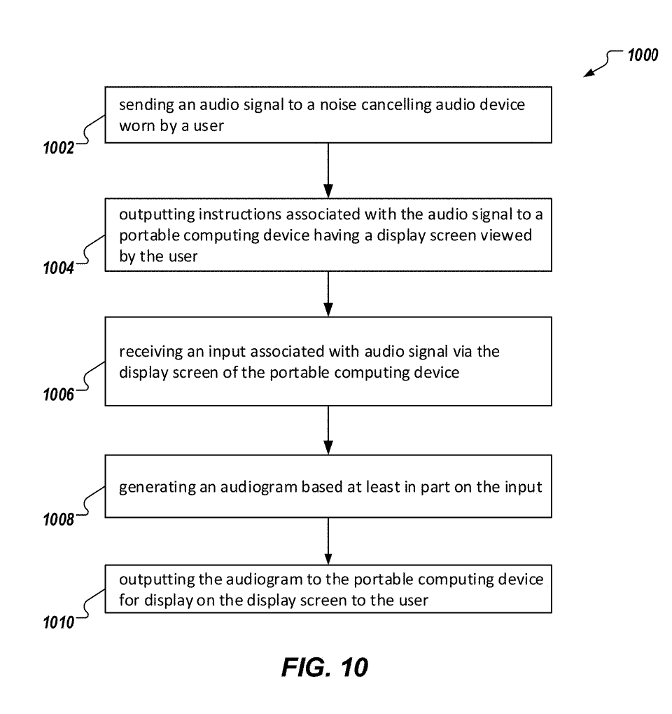

FIG. 10 depicts a flowchart of a process 1000 for carrying out a hearing test using the hearing assessment tool 200. The process 1000 includes sending an audio signal to a noise cancelling audio device (e.g., the audio device 112) worn by a user (1002). The process 1000 further includes outputting instructions (e.g., the instructions 702) associated with the audio signal to a portable computing device (e.g., the computing device 102) having a display screen (e.g., the display screen 116) viewed by the user (1004) and receiving an input associated with audio signal via the display screen of the portable computing device (1006). The process further includes generating an audiogram based at least in part on the input (1008) and outputting the audiogram to the portable computing device for display on the display screen to the user (1010).

Implementing the hearing assessment tool 200 with the portable audio device 112 and the portable computing device 102 can allow hearing test candidates to conveniently check their hearing outside of a clinical setting and potentially avoid consuming resources (e.g., time, money, and transportation) associated with clinical hearing tests in cases where results of hearing assessments carried out by the hearing assessment tool 200 indicate within normal limits. Furthermore, a patient can use his or her own audio device 112 and computing device 102 by downloading an app that implements the hearing assessment tool 200 on the computing device 102. Accordingly, hearing tests candidates can test their hearing periodically as desired to detect a hearing problem at a relatively early stage, as opposed to waiting until hearing becomes very poor to visit an audiologist. In this manner, the hearing assessment tool 200 can streamline hearing tests so that patients who really need to seek professional attention can do so, while others who do not exhibit medically significant hearing deterioration can avoid seeking such professional care.

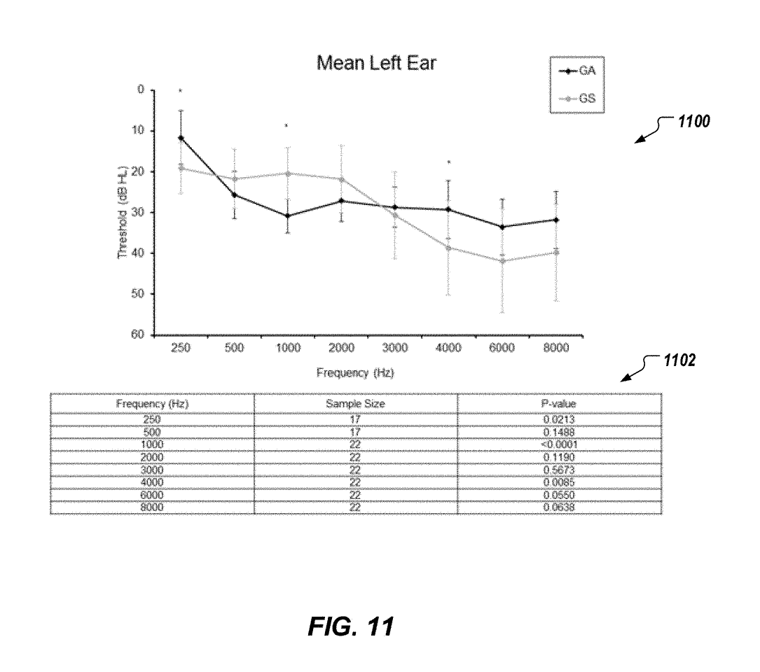

FIG. 11 depicts a graph 1100 that shows an audiogram (GA) generated using the hearing assessment tool 200 and an audiogram (GS) generated using a conventional, gold standard hearing assessment tool, as well as a set of data points 1102 associated with the graph 1100 for a subject's left ear.

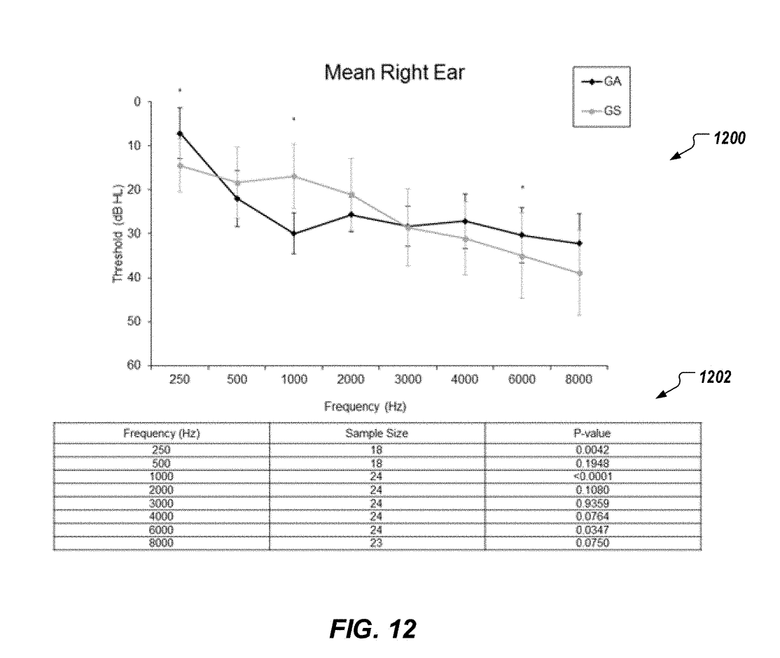

FIG. 12 depicts a graph 1200 that shows an audiogram (GA) generated using the hearing assessment tool 200 and an audiogram (GS) generated using a conventional, gold standard hearing assessment tool, as well as a set of data points 1202 associated with the graph 1200 for the subject's right ear.

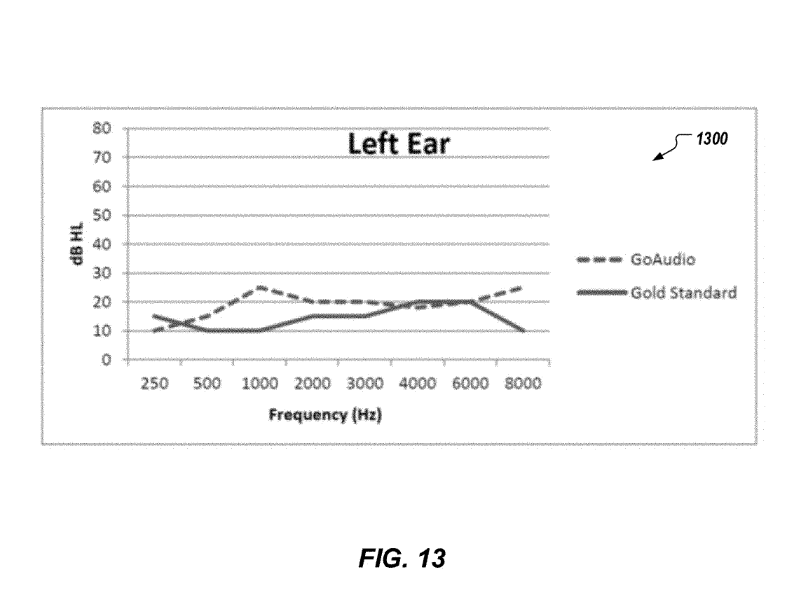

FIG. 13 depicts a graph 1300 that shows an audiogram (GoAudio) generated using the hearing assessment tool 200 and an audiogram (Gold Standard) generated using a conventional, gold standard hearing assessment tool for a subject's left ear.

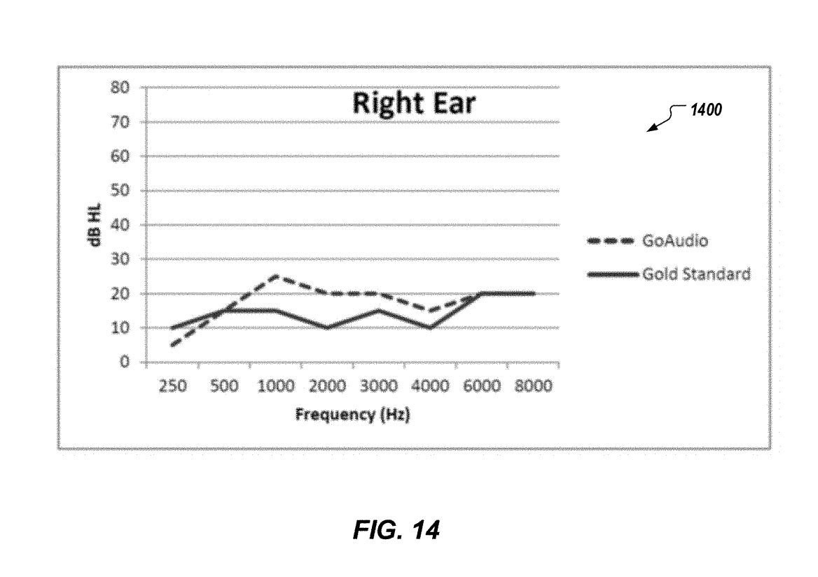

FIG. 14 depicts a graph 1400 that shows an audiogram (GoAudio) generated using the hearing assessment tool 200 and an audiogram (Gold Standard) generated using a conventional, gold standard hearing assessment tool for the subject's right ear.

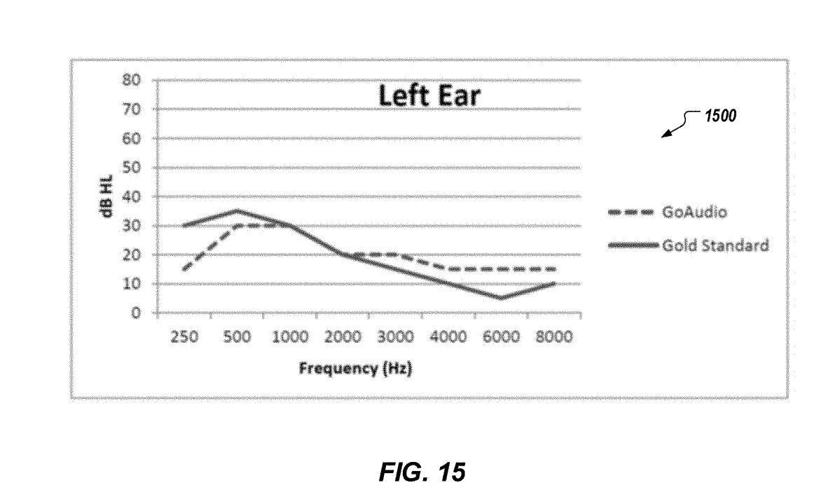

FIG. 15 depicts a graph 1500 that shows an audiogram (GoAudio) generated using the hearing assessment tool 200 and an audiogram (Gold Standard) generated using a conventional, gold standard hearing assessment tool for a subject's left ear.

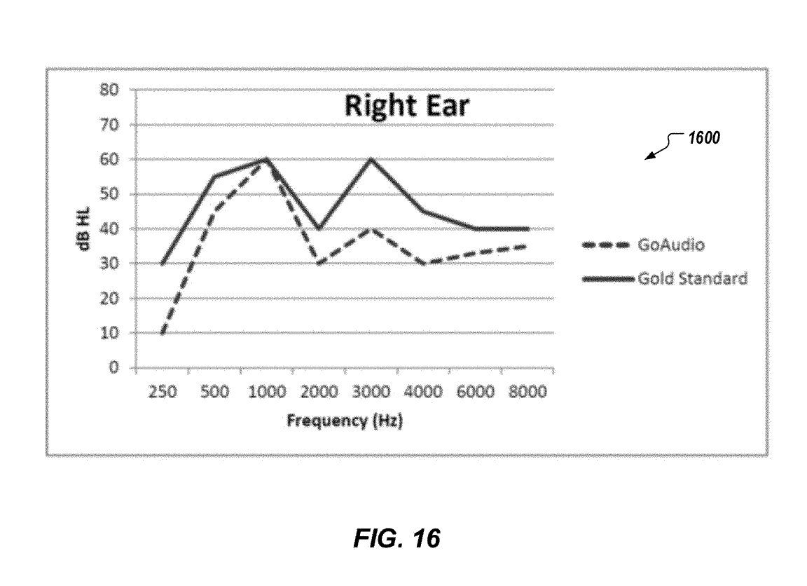

FIG. 16 depicts a graph 1600 that shows an audiogram (GoAudio) generated using the hearing assessment tool 200 and an audiogram (Gold Standard) generated using a conventional, gold standard hearing assessment tool for the subject's right ear.

A number of embodiments have been described. Nevertheless, it will be understood that various modifications may be made without departing from the spirit and scope of the present disclosure. Accordingly, other embodiments are within the scope of the following claims.

What is claimed is:

1. A method of executing a hearing test, comprising:
sending an audio signal to a noise cancelling audio device worn by a user;
outputting instructions associated with the audio signal to a portable computing device having a display screen viewed by the user;
receiving an input associated with audio signal via the display screen of the portable computing device;
generating an audiogram based at least in part on the input; and
outputting the audiogram to the portable computing device for display on the display screen to the user.

2. The method of claim 1, wherein sending the audio signal comprises sending a sound at a particular frequency and duration.

3. The method of claim 1, wherein the noise cancelling audio device comprises portable noise cancelling headphones.

4. The method of claim 1, further comprising storing the audiogram in an electronic medical record.

5. The method of claim 1, further comprising generating a description of a hearing assessment result based at least in part on the input and outputting the description to the portable computing device for display on the display screen to the user.

6. A hearing assessment system, comprising:
a noise cancelling audio device configured to be worn by a user;
a portable computing device having a display screen; and
executable instructions that configure the portable computing device to perform a method including:
sending a series of hearing assessment audio signals to the noise cancelling audio device;
outputting, on the display screen, instructions associated with the series of hearing assessment audio signals;
receiving inputs from the user in response to the series of hearing assessment audio signals;
generating an audiogram based on the inputs; and
outputting results of the audiogram on the display screen.
